(19) United States
(12) Patent Application Publication
(10) Pub. No.: US 2012/0135085 A1
(43) Pub. Date: May 31, 2012

(54) METHOD AND COMPOSITION FOR DISINFECTION AND PURIFICATION

(76) Inventors: ERNEST SCHMELTZER, Midland, TX (US); Brock Anderson, Lubboch, TX (US)

(21) Appl. No.: 13/305,099

(22) Filed: Nov. 28, 2011

Related U.S. Application Data
(60) Provisional application No. 61/417,483, filed on Nov. 29, 2010.

(51) Int. Cl. A61K 33/00; A61P 31/10; A61Q 11/00; A61P 17/06; A61P 31/12; A61P 31/22; A61P 17/10; A61P 17/02; A61P 37/00; A61K 8/19; A61P 31/04; A61P 17/12

(52) U.S. Cl. 424/600

(57) ABSTRACT

A method and composition for a non-toxic aqueous silicon solution.

### CROSS REFERENCE TO RELATED APPLICATIONS

This application claims the benefit of provisional application 61/417,483 filed Nov. 29, 2010 by the present inventors and the same is incorporated hereto in its entirety.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

1. Field of the Invention

The present invention relates generally to a method and composition for processing an aqueous silicon solution useful for disinfection and purification of a variety of surfaces and environments and further discloses novel and improved methods of use.

2. Description of the Related Art

Science is continually in search of a disinfectant or purifier that is effective, relatively inexpensive and non-toxic to healthy tissues or cells. In the realm of traditional medicine, antibiotics are the route of choice for treatment of infections, dental caries, infected burns and other types of bacterial infiltration. Although effective, antibiotics are accompanied by a host of unpleasant and sometimes harmful side effects. Furthermore, antibiotics are expensive, require intervention by a licensed medical practitioner, and are responsible for the mutation of bacteria into “superbugs” that are even more difficult to conquer.

Alternative choices embrace naturally occurring disinfectants and bugkillers such as colloidal silver and melaleuca (tea tree) oil. Efficacy of these has been questioned but there are other disadvantages as well. For example, the prolonged use of colloidal silver can cause a bluish tinge of the skin known as argyria and possibly neurological effects. Tea tree oil, although an effective deodorizer and disinfectant when used to treat topical conditions, is toxic when taken internally and so has limited application. Modern society is continually inflicted with immune disorders and airborne viruses that, increasingly, allopathic medicines are unable to control or cure.

U.S. Pat. No. 7,135,195 describes treating humans with a colorless silver colloidal composition. The composition describes very small colloidal silver particles in a solution and describes associated antimicrobial properties. Again, although such compositions in all likelihood possess some efficacy, the use of colloidal silver has questionable side effects including argyria.

Silicon is very basic, meaning it has a high pH. It has been found to have certain antimicrobial properties but is superior to other antimicrobial products due to its non-toxicity. In a 2004 Polish study surrounding endophthalmitis, silicon oil was found to have an antimicrobial function against Staphylococcus aureus, Pseudomonas aeruginosa and Candida albicans. Evaluation of antimicrobial properties of silicon oil-in vitro studes, Klin Oczna; 2004.

Silicon is present in biologic material as a silanate, an ether derivative of silicic acid. It is thought to play a role in the structure of glycosaminoglycans and their protein complexes. It is considered a vital trace mineral. Connective tissues including the aorta, trachea, tendons, bones, and skin all contain silicon. Silicon is necessary for the formation of collagen for bones and connective tissue. It promotes healthy nails, skin, and hair and calcium absorption in the early stages of bone formation.

Higher concentrations of silicon are naturally occurring in plants and in hard water, while lower concentrations are found in animal meat and soft water. Refining of food depletes the intrinsic silicon. Silicon additives are commonly used but are not well assimilated by the body. Unprocessed grains having high fiber have the highest concentrations of silicon.

Silicon is the second most available element found in earth's crust. It has been considered a “beauty mineral” for many years due to its role in connective tissue repair and preservation. It is said to assist with prevention of wrinkles and aging of skin. Although tablets containing silicon are sold on the market as supplements, the silicon in tablets can be difficult to assimilate, the majority of the silicon being excreted. An aqueous solution is preferable for both assimilation and ease of ingestion, however the few aqueous solutions on the market claiming health benefits have been found to have inconsistent concentrations and efficacies.

Part of the difficulty with commercial use of silicon is that historically there has not been a relatively fast way to process the silicon while simultaneously achieving a consistent, safe and reliable product with a high degree of purity. Another obstacle has been the difficulty in making the silicon available in a form that is easily usable or bioavailable for a variety of purposes, even including human or animal ingestion.

### SUMMARY OF THE DISCLOSED INVENTION

In accordance with the present disclosure, a method for processing and using an aqueous silicon solution useful for disinfection and purification of a variety of surfaces and solutions is disclosed. Methods for using the composition in a variety of useful applications are also disclosed.

It is an object of the disclosed invention to provide an effective and relatively fast way to process silicon that maintains purity and consistent Si concentrations.

It is an object of the disclosed invention to process a non-toxic composition that has antimicrobial properties.

It is an object of the disclosed invention to provide a method and composition for reducing or eliminating dental caries.

It is an object of the disclosed invention to provide a method and composition for reducing the microbial population on a burn and speed burn healing time.

It is an object of the disclosed invention to provide a method and composition for safely and effectively removing tattoos.

It is an object of the disclosed invention to provide a method and composition for purifying stagnant or contaminated water for human or animal ingestion.

It is an object of the disclosed invention to provide a method and composition for improving the hydration capabilities of water or sports drinks.

It an object of the disclosed invention to provide a method and composition for preventing and treating acne.

It is an object of the disclosed invention to provide a method and composition for treating and recovering sewer or otherwise contaminated water.

It is an object of the disclosed invention to provide a method and composition for eradicating fire ants and other undesired insects or bugs.

Other objects and advantages of the present invention will become apparent from the following descriptions, wherein, by way of example, an embodiment of the present invention is disclosed.

### BRIEF DESCRIPTION OF THE DRAWINGS

The figures or drawings contained herein represent preferred embodiments of the invention and are not intended to limit the scope. For a detailed description of various embodiments, reference will now be made to the accompanying illustrative drawings in which:

FIG. 1 depicts a table demonstrating various concentrations of Si achieved in the solution when varying processes were employed.

### DETAILED DISCLOSURE

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Specifically the present disclosure is a non-toxic aqueous silicon solution ranging from 2.5 to 5% silicon concentration, as well as the method of making it thereof that when employed as described results in a consistent and efficacious product for use in a variety of applications.

Essentially, the composition disclosed herein incorporates silicon, sodium hydroxide and water in a 6:1:10 ratio with an allowed variance of 6% on the silicon, 0.5% variance of sodium hydroxide and 8% variance on the water.

Disclosed herein is a composition, a method for processing said composition and methods for using the composition to achieve a variety of helpful uses. The disclosed method for processing the composition efficiently and consistently results in a stable high pH aqueous solution.

### Processing the Composition

The disclosed method for processing a stable high pH aqueous silicon solution useful for the purposes described herein incorporated the use of pretreated water free of impurities to reduce untested reactions and to improve consistency of the end product, overcoming the unreliable and inconsistent results abounding in prior known attempts. In the preferred embodiment, reverse osmosis water was used although other forms of reasonably pure water may be used as well. Testing is suggested to ensure that reaction times stay consistent.

In another effort to achieve a consistent end product, the water can be pretreated by delivering it through vortex tubes with 32 degree fins and further having the water spun to the left or counterclockwise as it exits the hose to the reaction vessel. The water may alternatively be treated by spinning it using flow tubes coiled in the proper direction, such as a single caduceus coil. Water treated in one of these ways is more conducive to a successful bioproduct and thus further ensures consistency of the end product. The sodium hydroxide can be delivered in a similar manner as the water. Other methods may be used if they yield a similarly stable and effective product.

In the preferred embodiment, in the reaction vessel, the water was preheated to 90-110 degrees prior to combining it with the Si and NaOH. This step surprisingly caused an acceleration in processing times without any concomitant adverse effects on the compounds or final product. The preheated water caused the reaction to both begin earlier and complete its cycle faster. The heat also caused a leaching of more Si into the solution, resulting in higher ppm or concentration of Si.

The reaction vessel can be of any configuration or geometric type with the preference being conical for the purpose of allowing the water to more conveniently cover the silicon although this configuration is not required with the methods employed herein. Cylinder shaped vessels are effective as well. Square or rectangular containers should be avoided due to the tendency of matter to collect in the sharp corners and produce eddys, preventing homogenous mixture of the contents and impeding the circulatory flow of the product. The reaction vessel may be glass or stainless steel. Other vessel materials may be used provided undue collateral reactions do not occur as a result of contact with the solution elements.

In the preferred embodiment, the silicon used was high grade silicon rock of at least 98% purity and was pre-etched through a previous exposure to water and sodium hydroxide. Pre-etching was found to enable a faster reaction and better final product due to the increased surface area being exposed. This is one aspect of accelerating the process and improving the consistency of the end product, both of which overcome disadvantages earlier discussed.

The NaOH used was a commercial grade which was the standard 50% dilution.

In the preferred embodiment, the Si, NaOH and pretreated heated water are combined in the reaction vessel in a ratio of 6:1:10, with some variances allowed, and are thoroughly mixed, preferably employing agitation or vibration to ensure effective mixing, to increase exposure to silicon surface areas and to further speed the reaction.

When using the described techniques, the reaction was deemed complete after 60-90 minutes of an obvious foam event. During the foam event, the volume in the reactor can increase up to three to four times the initial level when started. The reaction vessel should be chosen with this increase in volume anticipated to avoid overflow. The foam will be a light grey speckled with silicon particles. The liquid will become a dark grey as the silicon is forced into solution.

After the 60-90 minute waiting period to complete the reaction (significantly faster than any known process for creating a similar composition), the now aqueous solution may be filtered as required by the application. The solution is next exposed to natural or artificial UV light which stabilizes the new solution and allows the suspended nano-silicon particles to settle out.

During settling, the barrel or tote is again preferably vibrated to accelerate the settling process. The settling process is approximately 72 hours, after which the solution may be refiltered if desired.

Although during processing the solution is a dark grey color, the final liquid should be light amber in color and, for most purposes, have a ph ranging from 12.8-13.3. If it smells sweet, the ph is in good range; if sour or musty, the ph will be <12.5 ph. Viscosity is typically 1.22-1.34 SG (specific gravity) and blended with the left-spun water to the desired viscosity.

Total solution reaction time is from 2-8 hours, depending on the volume being produced. This is a far faster processing timeframe than described in any known prior art and is an important breakthrough . . . enabling commercial use for the first time as a result of the efficiency, efficacy and consistency of the process. When processed as described herein the silicon is also certified to be less than 0.001 percent lead or chromium.

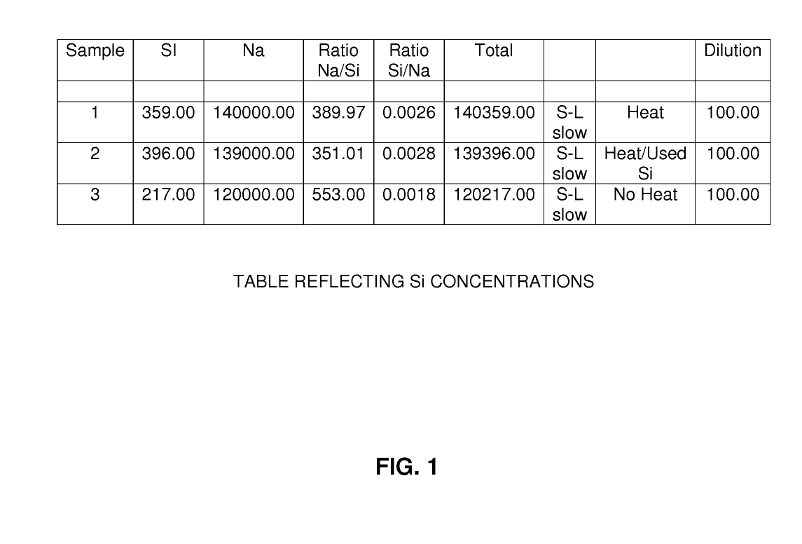

In FIG. 1 is demonstrated the increase in Si concentration achieved by adding heat to the Si processing steps, the concentration of Si increasing from 217 ppm to 359 ppm. An even more dramatic increase was noted when pre-etched or used Si was used in the processing rather than new Si, as shown by the increase to 396 ppm. The higher concentration of Si results in improved efficacy, bioavailability and efficiency. Consequently, the method and composition described herein achieves the described results quickly and safely.

### Applications of Use

The resulting non-toxic solution reduces microbial populations including bacteria, viruses, and fungi and can be used for a surprisingly diverse array of applications ranging from safely destroying dental caries, healing topical burns, purifying stagnate or contaminated water, treating sewage water, enabling water to be more easily utilized and absorbed by the cells when added to a water based hydration substance such as a sports beverage (due to reduced surface tension of the water), preventing and treating acne, cleaning, disinfecting, destroying mutated cells and eliminating harmful bacteria and other microorganisms. It appears to enhance the immune system and ward off disease, likely due to increasing the overall pH of the user.

The composition and methods disclosed herein also safely and non-toxically eradicate fire ants. Many types of insects are repelled by the composition, however plants thrive on it, making the composition an excellent vehicle for safe insect remediation for plants and lawns.

### Dental Caries

Dental caries occurs when bacterial processes destroy the hard enamel of the tooth. Bacteria naturally inhabit the mouth and create colonies that thrive on sugars and carbohydrates in the mouth. The lactic acid and acidic metabolites that the bacteria produce is deposited in the tooth, eventually demineralizing the tooth and causing dental caries, or cavities. The bacteria exist in oral biofilms, such as plaque, which builds up on the teeth between brushings. If left unchecked, the film builds up into a mineralized hard tartar. When the pH of the mouth drops into the acidic range, or below 5.5, the teeth lose minerals that maintain the hard protective enamel, and, concurrently, the saliva becomes more conducive to supporting excessive bacteria. The saliva normally protects our teeth, but if too many sugars and carbohydrates are consumed, the bacteria quickly build up plaque and tartar, to the extend that the saliva cannot penetrate the buildup effectively enough to stop demineralization and decay.

In live mammals, including humans, the majority of dental caries are eliminated by our normal saliva and masticulation processes. The high pH that the disclosed methods and compositions introduce onto the teeth when the solution is used as a mouthwash prevents the decay of teeth by depriving the bacteria of their desired more alkaline environment. The thin residual layer of silicon that remains after the solution is applied prevents further “footing” for the bacteria and thus prevents effective colonization on the tooth surface.

In testing, the residual silicone layer also prevented the tooth from being overly sensitive to hot, cold and sugars. The aqueous silicon solution offers protection to the teeth by creating a nano layer of silicon that the bacteria can't colinate on. In addition to creating a protective layer on the teeth, the aqueous silicon solution also dissolves current bacterial colonies (cavities). The method and composition disclosed herein literally dissolves the cavity from the tooth, leaving a hole or depression. As a result, the use of the composition also relieves the pain associated from the cavity. A 2% or slightly less concentration of the disclosed composition is preferably for oral solution.

The oral use of the disclosed aqueous silicon solution also heals sores in the mouth, gingivitis, halitosis and other oral conditions that include a bacterial component. By safely increasing the saliva pH, the disclosed aqueous silicon solution reduces the bacterial inhabitation and addresses the aforementioned issues.

Alcohol (the primary ingredient in mouthwashes) and hydrogen peroxide have been used to perform some of the functions described above but do not dissolve and remove cavities and are associated with unwanted side effects such as unnecessary drying of the mouth and destruction of healthy tissue and cells.

### Caries Removal Example:

An experiment was conducted using postmortem teeth infected with caries in order to validate the efficacy of the disclosed methods and compositions. The postmortem caries infected teeth were exposed to a 2% solution that was processed as described in the preceeding paragraphs. After dipping the teeth in the solution, the teeth appeared to have a shiny layer on them. Within seconds, the solution began to dissolve the caries. After letting the solution set for a few minutes, the tooth was brushed. The surface of the test tooth was at least 80% covered by dark dental caries. After the first brushing, the tooth surface showed only approximately 20% dark cavity. After a few more seconds and brushing the tooth showed less than 5% caries and was white and shiny in appearance.

Testing by the inventors has revealed that keeping the pH of the mouth over 7 prevents the occurrence of dental caries. Frequent rinsing of the mouth with this solution appears to have no harmful side effects and keeps the mouth and saliva at the desired pH to maintain healthy teeth and gums.

### Topical Burns

The disclosed aqueous silicon solution has been found in tests conducted by the inventors to increase the speed of the healing process when used on burns, cuts, insect bites, rashes, warts and psoriasis. When used topically in the concentrated form the user will note a quick sting followed by fast healing, reduction in scarring and a quick reduction in pain. This makes a practical addition to first aid kits. Larger burn areas should initially be treated with a 3% solution and increased as patient comfort allows. The solution disinfects the burn area and is effective at prevention infection from Staph aureus and methicillan resistant Staph aureus.

### Acne

As the disclosed aqueous silicon solution reduces the bacterial population on most surfaces and environments, yet is non-toxic, it can be used topically to prevent and treat acne. It can also be synergistically more effective at preventing acne when the topical use is accompanied by oral ingestion of the diluted solution to balance the overall body pH. It has also been shown to be beneficial in treating psoriasis, warts, insect bites, herpes and other skin conditions.

### Purifying Water

The disclosed aqueous silicon solution renders potable water from water that is otherwise stagnant and bacteria laden. Placed in watering troughs for livestock, the water will remain potable and oxygenated, protecting animal health. Used as a first aid/survival product, stagnant or contaminated water can be made potable as needed, whether during camping trips, foreign travel or use by the military. The concentration when used for this purpose should be a maximum of 5% concentration, with a 2.5 to 3% solution being preferred. Notably, mosquitoes and flies are repelled by the treated water.

### Superhydration

The disclosed aqueous silicon solution may be used to increase the hydration capabilities of beverages such as sports drinks or even water. The disclosed aqueous silicon solution, when added to a beverage, causes the cells to achieve increased uptake of the water or beverage by lowering the inherent surface tension.

### Disinfection

The disclosed aqueous silicon solution, due to its nontoxic and antimicrobial properties, is useful as a disinfectant for homes, institutions and restaurants. It reduces bacteria and other microbes while being biofriendly.

### Fruit and Vegetable Wash

Again, because of the non-toxic and antimicrobial properties inherent in the disclosed solution, it makes an ideal fruit and vegetable wash by safely removing such microbes as E. Coli and salmonella. Because the solution is non-toxic, a user may carry a small bottle with them and add drops to any food or beverages for which they are concerned, especially when traveling.

### Repelling Fire Ants or Other Undesirable Insects

When applied to a fire ant bed thoroughly, the ants were noted to completely evacuate the bed and did not return. This was achieved without damage or destruction to neighboring vegetation.

To employ an aqueous Si solution that is commercially viable it is critical that the process for making the solution be rapid, relatively inexpensive and non-toxic and that the solution be readily reproducible with consistent end products each time. This has not been accomplished until now.

While the disclosed methods and compositions have been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

It is to be understood that the embodiments disclosed herein are shown for illustrative purposes and are not intended to be construed as limitations of the disclosed method and system. Those skilled in the art will recognize or be able to ascertain in the course of routine experimentation, that variations and equivalents of the embodiments may be undertaken without departing from the scope of the invention.

Certain terms are used throughout the description to refer to particular method components. As one skilled in the art will appreciate, design and manufacturing companies may refer to a component by different names. This document does not intend to distinguish between components that differ in name but not function.

The terms “including” and “comprising” are used in an open-ended fashion, and thus should be interpreted to mean “including, but not limited to . . . . ” Also, the term “couple” or “couples” is intended to mean either an indirect or direct connection. Thus, if a first device couples to a second device, that connection may be through a direct connection or through an indirect connection via other intermediate devices and connections. Moreover, the term “method” means “one or more components” combined together. Thus, a method can comprise an “entire method” or “sub methods” within the method.

The use of the word “a” or “an” when used in conjunction with the word “comprising” may mean “one”, or may also mean “one or more.” The use of the term “or” in the claims is used to mean “and/or” unless explicitly indicated to refer to alternatives only or the alternatives are mutually exclusive, although the disclosures supports a definition that refers to only alternatives and “and/or.”

The methods and systems disclosed and claimed herein can be made and executed without undue experimentation based on the level of disclosure presented. While the methods and systems have been described in terms of their preferred embodiments, it will be apparent to those skilled in the art that they are not limited to the exact steps described and may vary from such description without departing from the scope and spirit of the invention. The substitutes and modifications employed by one skilled in the art are deemed to fall within the scope of the invention.

What is claimed is:

1. A non-toxic composition for reducing a microbial population, the composition comprising: an aqueous silicon solution ranging from 2.5 to 5% silicon concentration and further comprising H2O, Si and NaOH in a 6:1:10 ratio with an allowable variance of 6% on the silicon, 0.5% variance of sodium hydroxide and 8% variance on the water.

2. The composition of claim 1 wherein the aqueous silicon solution is processed by preheating water to a range of 90-110 degrees in a reaction vessel, adding preetched silicon rock of at least 98% purity, adding 50% dilution NaOH such that the ratio of the H2O, Si and NaOh is 6:1:10 with an allowable variance of 6% on the silicon, 0.5% variance of sodium hydroxide and 8% variance on the water, agitating the solution, allowing the solution to set for 60 to 90 minutes and exposing the solution to ultraviolet radiation.

3. The composition of claim 1 wherein the microbial population is bacteria.

4. The composition of claim 1 wherein the microbial population is viruses.

5. The composition of claim 1 wherein the microbial population is fungi.

6. A non-toxic method for reducing a microbial population, the method comprising: introducing into the microbial population an aqueous silicon solution ranging from 2.5 to 5% silicon concentration and further comprising H2O, Si and NaOH in a 6:1:10 ratio with an allowable variance of 6% on the silicon, 0.5% variance of sodium hydroxide and 8% variance on the water.

7. The method of claim 6 wherein the aqueous silicon solution is processed by preheating water to a range of 90-110 degrees in a reaction vessel, adding preetched silicon rock of at least 98% purity, adding 50% dilution NaOH such that the ratio of the H2O, Si and NaOh is 6:1:10 with an allowable variance of 6% on the silicon, 0.5% variance of sodium hydroxide and 8% variance on the water, agitating the solution, allowing the solution to set for 60 to 90 minutes and exposing the solution to ultraviolet radiation.

8. A non-toxic composition for reducing the symptoms of a condition suffered by a human or mammal suffering from the condition, the composition comprising: an aqueous silicon solution ranging from 2.5 to 5% silicon concentration and further comprising H2O, Si and NaOH in a 6:1:10 ratio with an allowable variance of 6% on the silicon, 0.5% variance of sodium hydroxide and 8% variance on the water.

9. The composition of claim 8 wherein the aqueous silicon solution is processed by preheating water to a range of 90-110 degrees in a reaction vessel, adding preetched silicon rock of at least 98% purity, adding 50% dilution NaOH such that the ratio of the H2O, Si and NaOh is 6:1:10 with an allowable variance of 6% on the silicon, 0.5% variance of sodium hydroxide and 8% variance on the water, agitating the solution, allowing the solution to set for 60 to 90 minutes and exposing the solution to ultraviolet radiation.

10. The composition of claim 8 wherein the condition is selected from the group consisting of dental caries, halitosis, gingivitis.

11. The composition of claim 8 wherein the condition is selected from the group consisting of psoriasis, warts, insect bites, herpes and acne.

12. The composition of claim 8 wherein the condition is burns.

13. The composition of claim 8 wherein the condition is dehydration.

14. The composition of claim 8 wherein the condition is compromised immune system.
